(19) United States
(12) Patent Application Publication
(10) Pub. No.: US 2024/0342433 A1
(43) Pub. Date: Oct. 17, 2024

(54) Vascular Probe Sensing System and Associated Delivery Device

(71) Applicant: Becton, Dickinson and Company, Franklin Lakes, NJ (US)

(72) Inventor: Jonathan Karl Burkholz, Salt Lake City, UT (US)

(21) Appl. No.: 18/634,015

(22) Filed: Apr. 12, 2024

Related U.S. Application Data
(60) Provisional application No. 63/459,085, filed on Apr. 13, 2023.

(51) Int. Cl. A61M 25/00; A61M 25/06; A61M 39/00; A61M 39/02; A61M 39/10

(52) U.S. Cl. CPC A61M 25/0097; A61M 25/0606; A61M 39/0247; A61M 39/1011; A61M 2039/0009; A61M 2039/0258; A61M 2039/0264; A61M 2039/0267; A61M 2039/0279; A61M 2039/1033; A61M 2039/1077; A61M 2205/3303; A61M 2205/3327; A61M 2205/3331; A61M 2205/3592; A61M 2205/502; A61M 2209/088; A61M 2230/20; A61M 2230/30

(57) ABSTRACT

A vascular access system includes a catheter assembly having a catheter and a near patient access port. A sensor probe delivery device is coupled to the near patient access port, with the delivery device including a sensing probe assembly having a probe member including one or more sensors and an electrical connector positioned proximally from the probe member. A housing of the delivery device movably receives the sensing probe assembly and includes a detachable portion separable from a remainder of the housing. A lock couples the housing to the near patient access port, and an advancement member may move relative to the housing to move the sensing probe assembly between a first position and a second position, with a distal end of the probe member disposed into or out past the catheter when in the second position to enable measuring of one or more blood-related parameters.

### CROSS-REFERENCE TO RELATED APPLICATION

The present application claims priority to U.S. Provisional Application No. 63/459,085 entitled “Vascular Probe Sensing System and Associated Delivery Device” filed Apr. 13, 2023, the entire disclosure of which is hereby incorporated by reference.

### BACKGROUND OF THE INVENTION

### Field of the Invention

The present invention is directed to a vascular access system and method of use thereof for measuring and monitoring blood-related parameters utilizing a sensing probe delivery device with an indwelling arterial catheter.

### Background of the Invention

Vascular access devices (VADs) are used in the medical field to access peripheral vasculature of a patient for purposes of infusion therapy and/or blood withdrawal. Common types of VADs include over-the-needle peripheral intravenous catheters (PIVCs), peripherally inserted central catheters (PICCs), central venous catheters (CVCs), and midline catheters. The VAD may be indwelling for short term (days), moderate term (weeks), or long term (months to years).

An exemplary use of a VAD as described above is in arterial catheterization. Arterial catheters and associated systems are used to continuously monitor and measure blood pressure, heart rate, and pulse contour to allow for immediate recognition of aberrant hemodynamic events and initiation of appropriate treatment. Arterial catheters are also used to provide samples for arterial blood for arterial blood gas (ABG) testing and analysis without the morbidity associated with repeat arterial puncture.

It is recognized that the use of current arterial catheters and associated arterial blood gas and blood sampling systems can be complicated and have numerous drawbacks, including blood exposure risk and other performance issues. Additionally, the existing hemodynamic monitoring and ABG collection systems can be very complicated, expensive, and require significant time and resources to collect ABG samples and maintain the arterial line to reduce the risk of complications, such as infection, CRBSI, proper line and device flushing, arterial blood preservation, etc.

Accordingly, a need exists for a system and device for facilitating improved arterial blood gas sampling and continuous pressure monitoring that overcomes the aforementioned limitations of existing systems and devices. The system would provide real time, continuous, or intermittent hemodynamic and blood parameter measurement and analysis, while eliminating the complexity and complication that come with the current systems and approaches.

### SUMMARY OF THE INVENTION

Provided herein is a vascular access system that includes a catheter assembly comprising a catheter and a near-patient access port, and a sensor probe delivery device coupleable to the near-patient access port to enable access to the catheter via the catheter assembly. The delivery device includes a sensing probe assembly having a probe member including one or more sensors configured to measure one or more blood-related parameters, the probe member having a distal end and a proximal end, and an electrical connector positioned proximally from the probe member, the electrical connector configured to operably connect to an external device. The delivery device also includes a housing configured to movably receive at least a portion of the sensing probe assembly within an inner volume thereof, the housing comprising a detachable portion separable from a remainder of the housing. The delivery device further includes a lock configured to couple the housing to the near patient access port and an advancement member configured to move relative to the housing to move the sensing probe assembly between a first position, in which a distal end of the probe member is disposed within the housing or the lock, and a second position in which a distal end of the probe member is disposed beyond the distal end portion of the housing and the lock and into the catheter or out past a distal tip of the catheter. With the sensing probe assembly in the second position, the probe member is positioned to measure the one or more blood-related parameters.

In some embodiments, the housing comprises a coupler provided at a distal end portion thereof, the coupler configured to mate with the lock or formed integrally with a portion of the lock, and wherein the detachable portion of the housing is configured to disconnect from the coupler.

In some embodiments, the detachable portion of the housing is configured to disconnect from the coupler via a twist-type disconnection.

In some embodiments, the detachable portion comprises a splittable housing including a pair of detachable housing portions that separate off from the coupler.

In some embodiments, the sensing probe assembly comprises a connector portion including a fitting configured to secure the proximal end of the probe member therein and a flange member joined with the fitting, wherein the connector portion is positioned adjacent the coupler when the sensing probe assembly is in the second position, with the connector portion retained in the coupler.

In some embodiments, the advancement member is coupled with the flange of the connector portion, such that movement of the advancement member relative to the housing causing a corresponding movement of the sensing probe assembly.

In some embodiments, the electrical connector is joined to the connector portion and extends out proximally therefrom.

In some embodiments, the electrical connector is positioned within the housing when the sensing probe assembly is in each of the first position and the second position, with the electrical connector being accessible when the sensing probe assembly is in the second, upon disconnecting of the detachable portion.

In some embodiments, the sensing probe assembly comprises a wired extension joined to the connector portion and extending out proximally therefrom, and wherein the electrical connector is coupled to the wired extension at a proximal end thereof.

In some embodiments, with the electrical connector coupled to the proximal end of the wired extension, the electrical connector is spaced apart from the connector portion and the coupler when the sensing probe assembly is in the second position.

In some embodiments, a wireless module is coupled to the electrical connector, with the wireless module configured to receive the one or more blood-related parameters from the sensing probe assembly and analyze and display the one or more blood-related parameters and/or wirelessly transmit the one or more blood-related parameters to a processing device.

In some embodiments, a patient data cable is coupled to the electrical connector and configured to transmit the one or more blood-related parameters to a processing device.

In some embodiments, the catheter assembly comprises a catheter adapter coupled to a proximal end of the catheter, the catheter adapter comprising an adapter port, and a connector connected to the adapter port via an extension tube, wherein the near patient access port is provided at a proximal end of the connector.

Also provided is a method of using a vascular access system, with the method including coupling a sensor probe delivery device to the near-patient access port of the catheter assembly, advancing the sensor probe assembly from the first position to the second position such that the distal end of the probe member extends into the catheter or beyond the distal end of the catheter, disconnecting the detachable portion of the housing, and connecting an external device to the electrical connector of the sensor probe assembly, the external device comprising one of a wireless module and a data cable.

In some embodiments, the method includes securing the wireless module in place via one or more of a stabilizing platform and a securement dressing.

Also provided is a sensor probe delivery device coupleable to a near-patient access port of a catheter assembly. The delivery device includes a sensing probe assembly comprising a probe member including one or more sensors configured to measure one or more blood-related parameters, the probe member having a distal end and a proximal end, and an electrical connector positioned proximally from the probe member, the electrical connector configured to operably connect to an external device. The delivery device also includes a housing configured to movably receive at least a portion of the sensing probe assembly within an inner volume thereof, the housing comprising a detachable portion separable from a remainder of the housing, a lock configured to configured to be connected to the near patient access port, and an advancement member configured to move relative to the housing to move the sensing probe assembly between a first position, in which a distal end of the probe member is disposed within the housing or the lock, and a second position in which a distal end of the probe member is disposed beyond the distal end portion of the housing and the lock. With the sensing probe assembly in the second position, the probe member is positioned to measure the one or more blood-related parameters.

In some embodiments, the housing includes a coupler provided at a distal end portion thereof, the coupler configured to mate with the lock or formed integrally with a portion of the lock, and wherein the detachable portion of the housing is configured to disconnect from the coupler.

In some embodiments, the sensor probe delivery device includes a connector portion having a fitting configured to secure the proximal end of the probe member therein and a flange member joined with the fitting, wherein the connector portion is positioned adjacent the coupler when the sensing probe assembly is in the second position, with the connector portion retained in the coupler.

In some embodiments, the advancement member is coupled with the flange of the connector portion, such that movement of the advancement member relative to the housing causing a corresponding movement of the sensing probe assembly.

In some embodiments, the electrical connector is joined to the connector portion and extends out proximally therefrom, with the electrical connector positioned within the housing when the sensing probe assembly is in each of the first position and the second position, and with the electrical connector being accessible when the sensing probe assembly is in the second, upon disconnecting of the detachable portion.

### BRIEF DESCRIPTION OF THE DRAWINGS

FIG. 1 is a top view of an arterial access system including a sensor probe deliver device, in accordance with an aspect of the disclosure;

FIG. 2 is a top view of the sensor probe delivery device of the arterial access system of FIG. 1, in accordance with an aspect of the disclosure;

FIG. 3 is an exploded view of the sensor probe delivery device of FIG. 2;

FIG. 4 is a top view of the sensor probe delivery device of FIG. 2, showing a detachable housing portion, in accordance with an aspect of the disclosure;

FIG. 5 is a top view of the sensor probe delivery device of FIG. 2, showing a detachable housing portion, in accordance with an aspect of the disclosure;

FIG. 6 is a top view of the sensor probe delivery device of FIG. 2, showing a detachable housing portion, in accordance with an aspect of the disclosure;

FIG. 7 is a top view of a sensor probe assembly included in the sensor probe delivery device of FIG. 2, in accordance with an aspect of the disclosure;

FIG. 8 is a side cross-sectional view of a distal portion of a probe member of the sensor probe assembly of FIG. 7, in accordance with an aspect of the disclosure;

FIG. 9 is a side cross-sectional view of a distal portion of a probe member of the sensor probe assembly of FIG. 7, in accordance with an aspect of the disclosure;

FIG. 10 is a side cross-sectional view of the sensor probe delivery device of FIG. 2, showing the sensor probe in a first, retracted position;

FIG. 11 is a side cross-sectional view of the sensor probe delivery device of FIG. 2, showing the sensor probe assembly in a second, extended position;

FIGS. 12-14 illustrate a process flow for interfacing a sensor probe delivery device with a catheter assembly for measuring and analyzing one or more blood-related and hemodynamic parameters, in accordance with an aspect of the disclosure;

FIG. 15 is a schematic view of a wireless board unit useable with the sensor probe delivery device of FIG. 2, in accordance with an aspect of the disclosure;

FIG. 16 is a schematic view of a communication system useable with the wireless board unit of FIG. 15, in accordance with an aspect of the disclosure;

FIG. 17 is a top view of a sensor probe delivery device useable with the arterial access system of FIG. 1, with the sensor probe delivery device in a first configuration, in accordance with an aspect of the disclosure;

FIG. 18 is a top view of the sensor probe delivery device of FIG. 17, with the sensor probe delivery device in a second configuration;

FIG. 19 is a top view of the sensor probe delivery device of FIG. 17, with the sensor probe delivery device in the second configuration and connected to a catheter assembly and a wireless board unit, in accordance with an aspect of the disclosure;

FIG. 20 is a top view of the sensor probe delivery device of FIG. 17, with the sensor probe delivery device in the second configuration and connected to a catheter assembly and a patient data cable, in accordance with an aspect of the disclosure; and

FIG. 21 is a top view of a sensor probe delivery device useable with the arterial access system of FIG. 1, in accordance with an aspect of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device (e.g., the end of the device being manipulated by the user) would be the proximal end of the device.

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

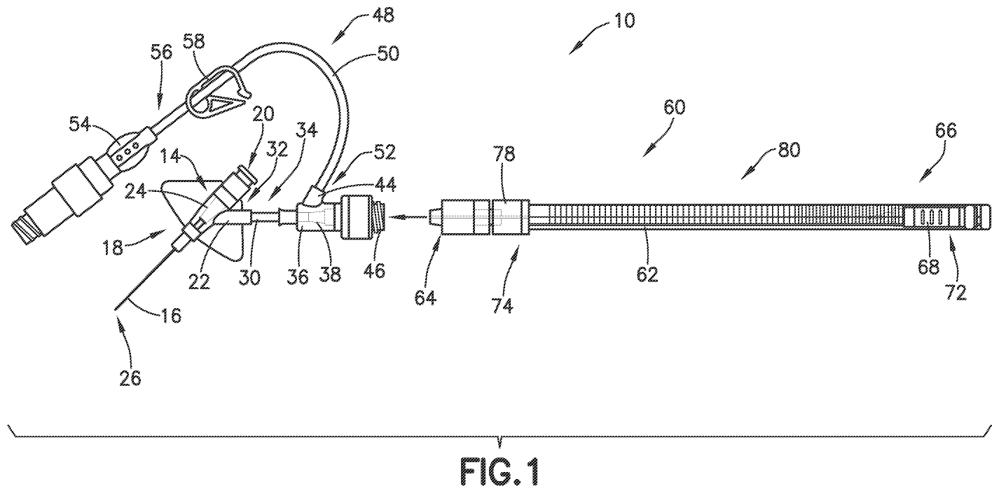

Referring to FIG. 1, shown is a non-limiting embodiment of a vascular access system 10 for facilitating blood-related parameter and hemodynamic analysis and monitoring. While the vascular access system 10 is described hereafter as an arterial access system that facilitates arterial blood gas (ABG) testing and pressure monitoring, it is recognized that the system could also be utilized for measuring and monitoring bloodstream-based parameters in a vein or other portion of the vasculature.

As shown in FIG. 1, arterial access system 10 may include a catheter assembly that provides access to the vasculature of a patient. According to embodiments of the disclosure, the catheter assembly 12 may have any of a number of suitable configurations, including being constructed/arranged as an integrated catheter assembly or a non-integrated catheter assembly that includes an extension set with a near-patient access port. Thus while FIG. 1 illustrates an integrated catheter assembly 12, it is recognized that aspects of the disclosure may be directed to other catheter assemblies. In FIG. 1, the catheter assembly 12 includes a catheter adapter 14 and associated catheter 16. The catheter adapter 14 may include a distal end 18 and a proximal end 20. In some embodiments, the catheter adapter 14 may include an additional adapter port 22 that may disposed between the distal end 18 and the proximal end 20 or disposed at the proximal end 20. The catheter adapter 14 may include a first lumen 24 extending through the distal end 18 and the proximal end 20, and the first lumen 24 may be sealed at proximal end 20 of catheter adapter 14. The catheter 16 extends from the distal end 18 of catheter adapter 14 and may be configured as an arterial catheter that is placed into an artery of the patient, with a distal end or tip 26 of the catheter 16 positioned appropriately within an artery (or other vein) to enable a blood draw from the patient. In some embodiments, the catheter 16 may be inserted into an artery such that the distal tip 26 (an opening therein) faces upstream and into the arterial blood flow. Catheter 16 may be formed of any suitable material and may be of any useful length, as known to those of skill in the art.

In some non-limiting embodiments or aspects, the catheter assembly 12 may include a first fluid conduit 30 extending from the port 22. First fluid conduit 30 may be formed of any suitable material known to those of skill in the art and may have a distal end 32 and a proximal end 34. The distal end 32 of first fluid conduit 30 is coupled to port 22, while the proximal end 34 of first fluid conduit 30 may be coupled to a connector 36. Connector 36 may be a t-connector (e.g., one side port arranged at a 90 degree angle relative to a longitudinal axis of connector 36), a y-connector (e.g., one side port arranged at a 15-85 degree angle relative to a longitudinal axis of connector 36), or any other type of connector known in the art. The connector 36 includes a second lumen 38 therethrough, having any number of branches suitable for the type of connector, such as a branch extending between distal and proximal ends of connector 36 and a branch provided to a port 44 of the connector 36.

In some non-limiting embodiments or aspects, catheter assembly 12 may include a needleless access connector 46 coupled to the proximal end of connector 36, with the needleless access connector 46 providing a near-patient access port to the catheter assembly 12. The needleless access connector 46 may be configured as a split-septum connector or self-healing septum connector, as examples.

In some non-limiting embodiments or aspects, catheter assembly 12 may also include an extension set 48 coupled to the port 44 of the connector 36. The extension set 48 may include a second fluid conduit 50 coupled to port 44 at end 52 of the conduit 50 and a luer connection 54 (e.g., needleless access connector) at opposing end 56, with a clamp 58 provided on second fluid conduit 50 that allows for occlusion thereof. The extension set 48 may be used to provide a fluid path to or from the catheter assembly 12 to enable fluid or medication delivery, blood aspiration, or to an ex-vivo hemodynamic monitoring device that monitors blood pressure, heart rate, and/or pulse contour of a patient, based on arterial blood drawn through the catheter assembly 12 (and through extension set 48).

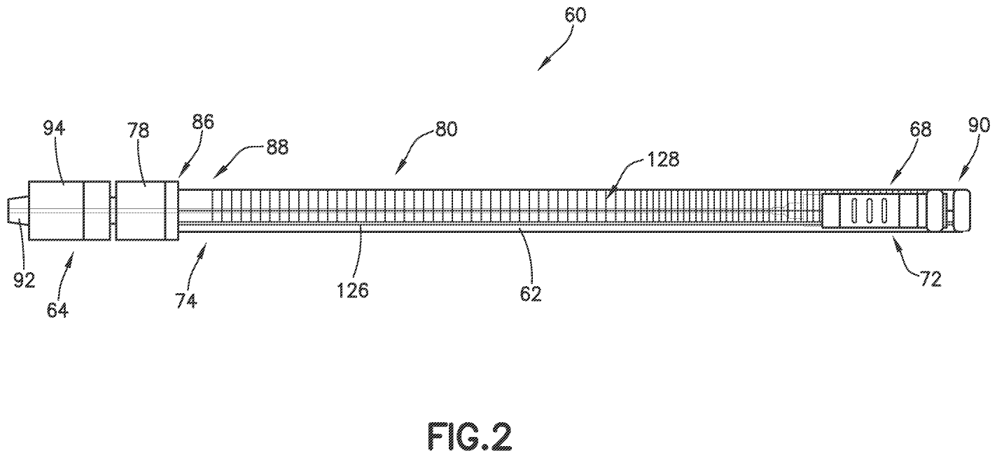

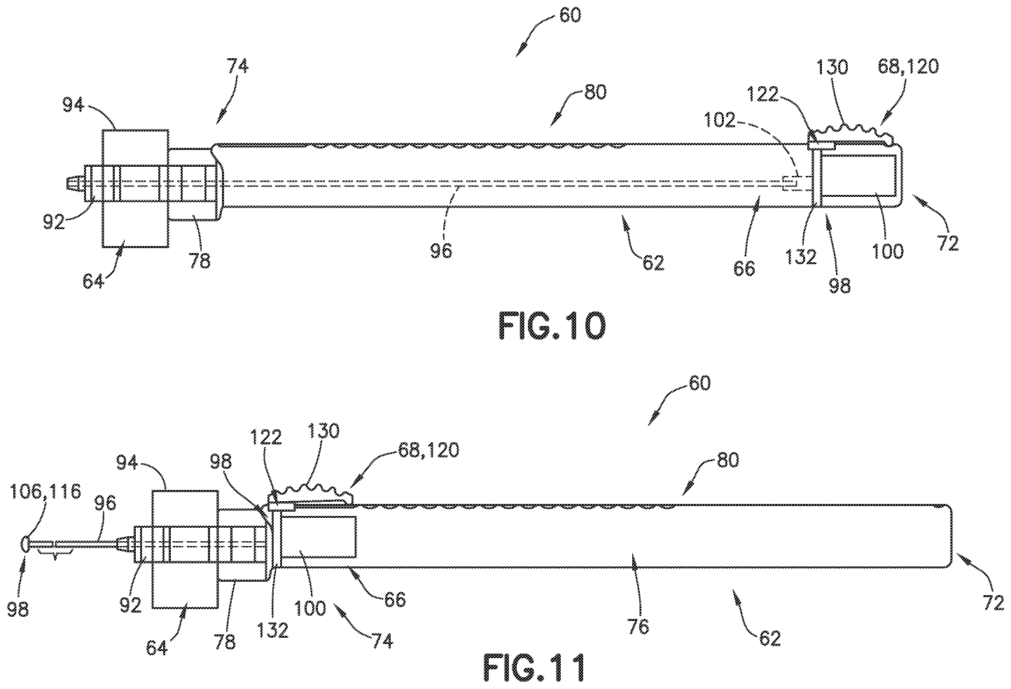

According to aspects of the disclosure, arterial access system 10 further includes a delivery device 60 that may be operated to introduce a sensing probe into the vasculature of the patient, with such a sensing probe enabling blood gas analysis for example. As shown in FIG. 1 and in further detail in FIGS. 2-11, according to a non-limiting embodiment, the delivery device 60 includes at least a housing 62, a coupling device or “lock” 64, a sensing probe assembly 66, and an advancement member 68. As will be described in further detail below, the sensing probe assembly 66 is moveable within the housing 62 so as to provide for advancement of a portion (i.e., a probe member) of the sensing probe assembly 66 from a first or retracted position inside the housing 62 (FIG. 10) to a second or advanced position outside of the housing 62 (FIG. 11), such that a distal end thereof may be routed into the catheter assembly 12. Once a portion of the sensing probe assembly 66 has been routed into the catheter assembly 12 and into or out past the indwelling catheter 16, a portion of the housing 62 may be removed, such that the sensing probe assembly 66 may remain in place for a period of time without interference from the housing 62.

As shown in FIGS. 2-5, the housing 62 of delivery device 60 can be an elongate member having a proximal end 72 and a distal end 74 and defining an inner volume 76. The housing 62 generally includes a coupler 78 provided at the distal end 74 and a detachable portion 80 positioned proximally from the coupler 78. The coupler 78 is configured to provide for connection of the housing 62 to lock 64, while the detachable portion 80 is configured to selectively disconnect from coupler 78. In some embodiments, the housing 62 may include one or more features or surface finishes on an outer surface thereof that can be arranged to increase the ergonomics of the delivery device 60, which in some instances can allow a user to manipulate the delivery device 60 with one hand (i.e., single-handed use).

The coupler 78 can be any suitable shape, size, and/or configuration and may be integrated with lock 64 or configured for connection thereof to lock 64. In some embodiments, the coupler 78 may include a set of threads 82 that enable a threaded coupling with an associated threaded portion 84 of the lock 64. In other embodiments, the coupler 78 may be integrally formed with at least part of lock 64. The coupler 78 may further include a mating feature 86 on a proximal end thereof that provides for engagement between the coupler 78 and the detachable portion 80 of housing 62. According to embodiments, the mating feature 86 may be in the form of a groove provided about an outer circumference/perimeter of the coupler 78 with which an end of the detachable portion 80 may engage, or may be in the form of a threaded connection with which an end of the detachable portion 80 may engage.

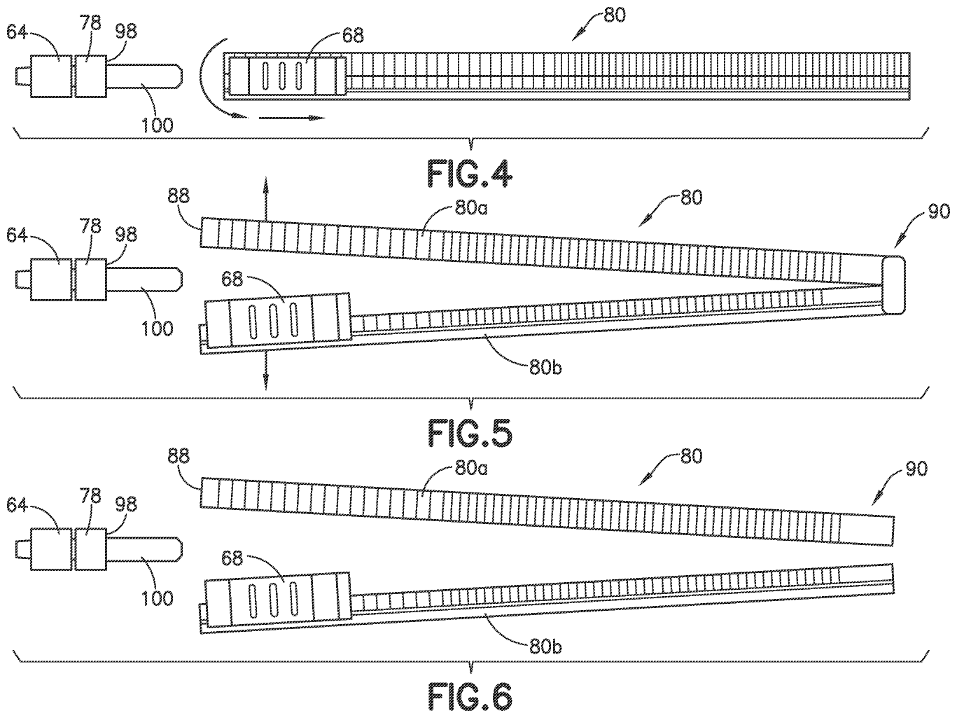

According to embodiments, detachable portion 80 of housing 62 is configured so as to disconnect from coupler 78 responsive to actuation of the detachable portion 80. Referring to FIGS. 4-6, various embodiments of detachable portion 80 are shown that provide for disconnection of detachable portion 80 relative to coupler 78. In an embodiment shown in FIG. 4, the detachable portion 80 is configured to engage/disengage with coupler 78 via a twist type engagement, with a threaded connection or other twist-type connection at the distal end 88 of detachable portion 80 engaging with mating feature 86 of coupler 78. In embodiments shown in FIGS. 5 and 6, the detachable portion 80 is configured to as a splittable housing formed of a pair of detachable housing portions 80a, 80b that may be selectively detached from each other, with the detachable housing portions 80a, 80b able to disengage from mating feature 86 of coupler 78 when separated apart. In the embodiment of FIG. 5, the detachable housing portions 80a, 80b may remain joined at a proximal end 90 thereof when split apart at their distal end 88, while in the embodiment of FIG. 6 the detachable housing portions 80a, 80b are provided as entirely separate components that may completely separate from one another.

While FIGS. 4-6 illustrate a detachable portion 80 of housing 62 disconnecting from coupler 78, it is recognized that other portions of the housing 62 (or delivery device 60) may be removably coupled to any components that remain with the patient (i.e., sensing probe assembly 66 and other possible components), so long as at least a portion of the delivery device 60 may be detached to reduce the size/footprint of the components that remain with the patient. As one non-limiting example, the coupler 78 may be configured to quickly detach/separate from lock 64.

Referring again now to FIGS. 1-6, the lock 64 of delivery device 60 is provided at the distal end 74 of the housing 62, with the lock 64 providing for reversible coupling of the delivery device 60 to catheter assembly 12, such as via needleless access connector 46 as shown in FIG. 1. In some embodiments, the lock 64 is configured as a male luer connector that mates with a female luer connector of needleless access connector 46, with the male luer connector of lock 64 having an elongated member 92 surrounded by a rotating collar 94. The rotating collar 94 may be rotated to threadingly engage the male luer connector to the female luer connector of needleless access connector 46. According to embodiments, the lock 64 may be provided as an entirely separate component from housing 62, with a proximal end thereof configured to engage with the coupler 78 of housing 62, with one embodiment having the proximal end of lock 64 configured as a threaded protrusion 84 that selectively engages a set of threads 82 on the coupler 78. In other embodiments. the elongated member 92 of lock may be formed integrally with the housing 62 (i.e., with the coupler 78) and extend out distally therefrom, with the rotating collar 94 secured about the elongated member 92 and being rotatable relative thereto. While lock 64 is structured as a male luer connector in the illustrated embodiment, it is appreciated that alternative embodiments of delivery device 60 may include a lock 64 of another type to secure delivery device 60 to catheter assembly 12, including clips, blunt plastic cannulae, blunt metal cannulae, hybrid luers (e.g., with a cannula) friction fits, and the like.

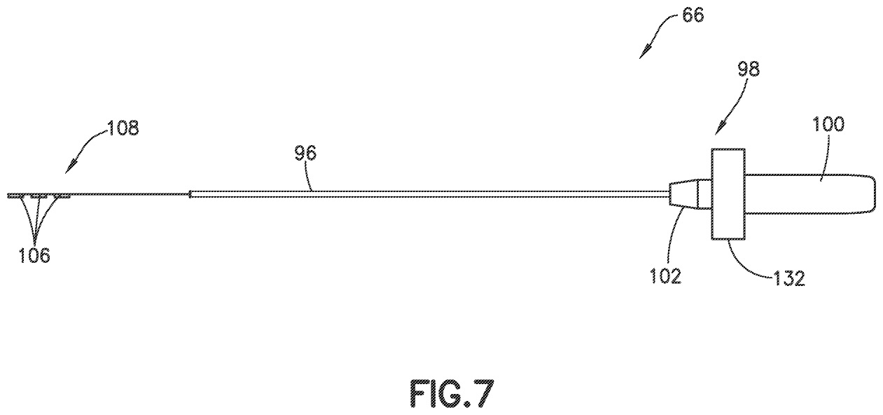

As indicated above, the sensing probe assembly 66 is moveably received within the housing 62 so as to provide for advancement of the sensing probe assembly 66 relative to the housing 62. According to embodiments, and as best shown in FIG. 7, the sensing probe assembly generally includes a sensing probe member 96 (“probe member 96”), a connector portion 98, and an electrical connector 100. The probe member 96 may be coupled to connector portion 98 via a fitting 102 of connector portion 98 and, in the illustrated embodiment, electrical connector 100 is also coupled to connector portion 98. The electrical connector 100 is provided at a proximal end of the sensing probe assembly 66 and provides an output port for the sensing probe assembly 66 to which an external device/component may be connected. According to embodiments, the electrical connector 100 is configured to enable attachment thereof of a patient data cable or a Wi-Fi enabled wireless board with processor, communications, power, sensor leads, and optional display, as explained in further detail below.

The probe member 96 is sized to enable introduction thereof into the fluid path (i.e., into a lumen of catheter 16, lumen 24 of catheter adapter 14, and first fluid conduit 30) of catheter assembly 12 and for advancement therethrough. Accordingly, the probe member 96 can have an outer diameter (e.g., between a 10-gauge and a 30-gauge) that is smaller than the smallest lumen of the catheter assembly fluid path. The probe member 96 can have a length that is sufficient to place a distal end 98 of the probe member 96 in a desired position within the fluid path of the arterial access system 10. Thus, in one embodiment, the probe member 96 may have a length sufficient to provide for advancement of the distal end 98 thereof out from the housing 62 and through the catheter assembly (i.e., through connector 36, fluid conduit 30, catheter adapter 14 and catheter 16), and all the way out past the distal tip 26 of catheter 16.

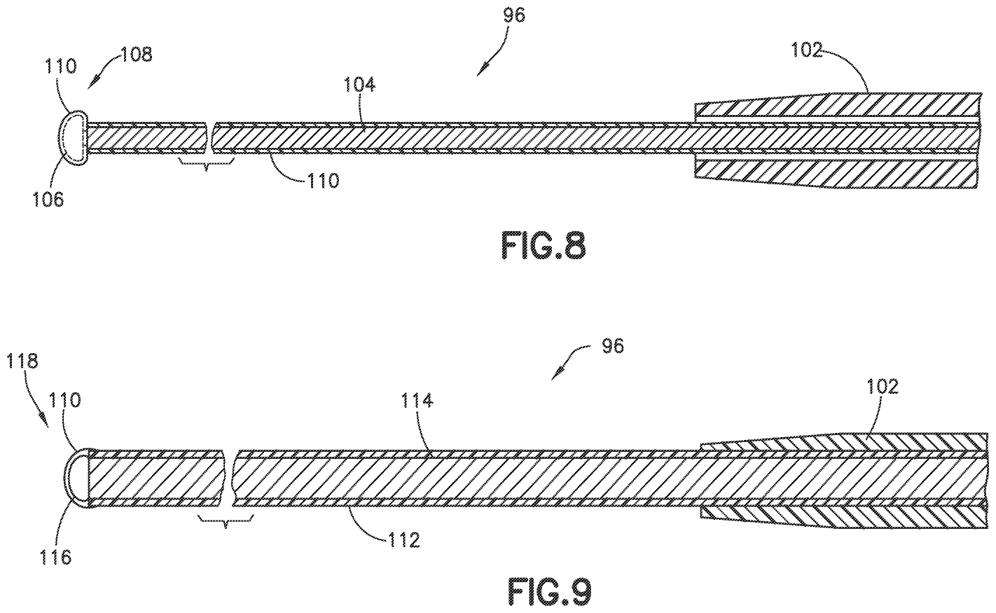

Illustrative embodiments of probe member 96 are provided in FIGS. 8 and 9, according to non-limiting embodiments. Referring first to FIG. 8, in one embodiment, the probe member 96 generally includes a support wire 104 and one or more sensors 106 (i.e., a sensor or sensor bundle) that is/are coupled to (or near) a distal tip 108 of the support wire 104. The support wire 104 may constructed of metal (e.g., nitinol) or other suitable material and is formed to have a thin profile such that the wire has some flexibility. The sensor or sensor bundle 106 is configured to monitor various vital signs or physiological parameters of a patient, referred to hereafter generally as “blood-related parameters”, such as blood pressure (venous or arterial), blood gases, pH, electrolytes, temperature, oxygen level, or other targeted physiological or procedural parameters, and may be configured to wirelessly transmit measurements of such parameters to a remote receiver.

According to embodiments, each of the support wire 104 and sensor(s) 106 may be coated with an antithrombogenic coating 110. Suitable antithrombogenic materials for use in medical devices that access the vasculature are known to those of skill in the art and may include, without limitation, heparin, heparin-mimetic materials, heparin, albumin, hydrophobic lubricants, fluorinated lubricants, compositions including fluoro-containing functional moieties, silicone-containing functional moieties, and/or poly (ethylene glycol) (PEG) functional moieties, antithrombogenic polymers (e.g., those containing one or both of poly(ethylene oxide) (PEO)/sulfate/sulfonate and poly(propylene oxide) (PPO), poly(2-methoxyethyl acrylate)), and combinations, phosphorylcholine, pro-drugs, and derivatives thereof. The antithrombogenic coating 110 may be applied in the form of a film and/or a coating on one or more of support wire 104 and sensor(s) 106 that allows for the antithrombogenic coating to be released upon being exposed to one or more enzymes in the blood, as the probe member 96 is received within catheter 16 of catheter assembly 12. The antithrombogenic coating 110 acts to prevent blood clots from building on the surface of the probe member 96, and specifically the sensor(s) 106, as over time such blood clots detrimentally affect performance of the sensor(s) 106. Application of the antithrombogenic coating 110 to the sensor(s) 106 can thus extend the useful life of the probe 96.

Referring now to FIG. 9, in another embodiment, probe member 96 generally includes a protective outer tubing 112, an optical fiber 114 housed within the outer tubing 112, and an optical sensor 116 that is coupled to a distal tip 118 of the optical fiber 114 and tubing 112. The outer tubing 112 and optical fiber 114 form a flexible member that provides for bending of the probe member 96 as it is inserted into the vasculature of a patient through catheter 16. The sensor 116 is configured to acquire measurements of vital signs or other physiological parameters as part of a diagnostic test and/or over a prolonged, continuous period of time, and may be configured to wirelessly transmit measurements of such parameters to a remote receiver.

According to embodiments, the distal tip 118 of the outer tubing 112 and the sensor 116 are coated with an antithrombogenic coating 110. As described above with regard to the probe member 96 of FIG. 8, the antithrombogenic coating 110 may be released upon being exposed to one or more enzymes in the blood, as the probe member 96 is received within catheter assembly 10. The antithrombogenic coating 110 acts to prevent blood clots from building on the surface of the probe member 96, and specifically the sensor 116, as over time such blood clots detrimentally affect performance of the sensor 116. Application of the antithrombogenic coating to the sensor 116 can thus improve diagnostic or continuous measurement capability of the sensor 116 (by preventing thrombus attachment or fouling of the sensor 116) and extend the useful life of the probe member 96.

Referring again now to FIGS. 1-11, and in particular to FIGS. 2, 3, 10, and 11, in accordance with one aspect of the disclosure, advancement member 68 of delivery device 60 includes a first portion 120 and a second portion 122. The first portion 120 is movably disposed along an upper surface 128 of the housing 62 and the second portion 122 is movably disposed within the inner volume 76 of the housing 62. In some embodiments, the arrangement of the advancement member 68 and the housing 62 is such that a connecting portion 124 of the advancement member 68 that joins the first and second portions 120, 122 is seated within a slot 126 formed in the upper surface 128 of the housing 62—the slot 126 generally extending between the proximal and distal ends 72, 74 of the housing 62 (i.e., of the detachable portion 80). As the first and second portions 120, 122 are joined together, movement of the first portion 120 along the upper surface 128 of the housing 62 results in a corresponding movement of the second portion 122 within the inner volume 76.

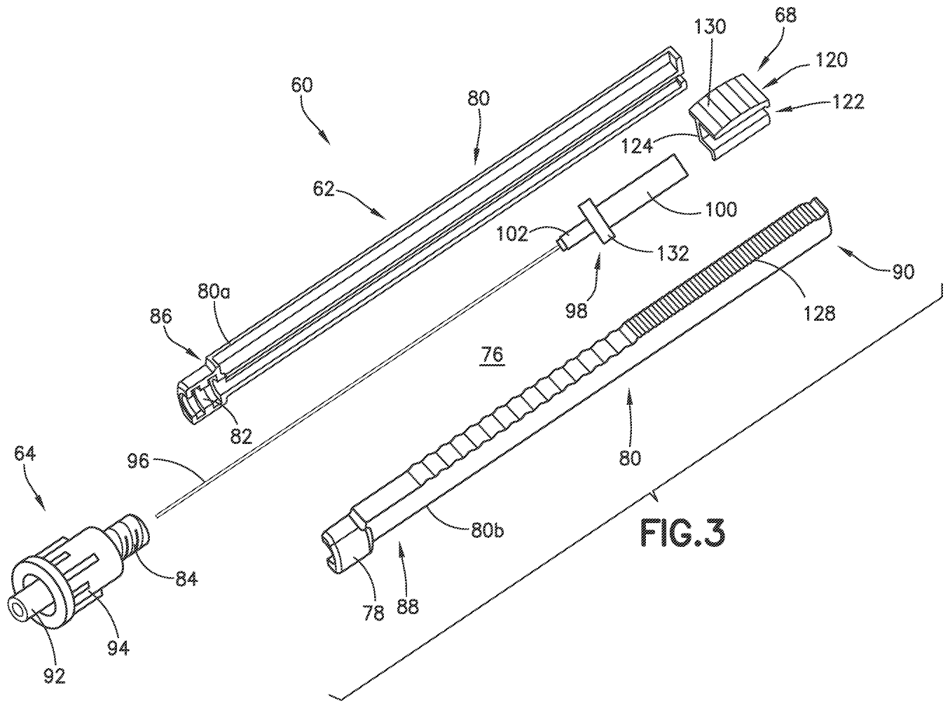

As shown in FIGS. 2 and 3, the first portion 120 of the advancement member 68 may be configured as a tab 130 having a top side engageable by a user and an underside that is in contact with the upper surface 128 of the housing 62. In such embodiments, the upper surface 128 of the housing 62 can include a track, for example, a set of ribs, ridges, bumps, grooves, and/or the like along which the underside of tab advances when the advancement member 68 is engaged by a user. In this manner, a user can engage the first portion 120 of the advancement member 68 and can move the advancement member 68 relative to the housing 62.

As shown in FIGS. 3, 10 and 11, the second portion 122 is configured to engage with the sensing probe assembly 66 to grip or retain a portion of the sensing probe assembly 66. More particularly, the second portion 122 of advancement member 68 may engage the sensing probe assembly 66 at or adjacent the connector portion 98 thereof, such as by engaging a flange 132 or protrusion provided on connector portion 98, between the fitting 102 and electrical connector 100. Due to a portion of the sensing probe assembly 66 being secured to second portion 122, movement of the advancement member 68 relative to housing 62 causes a corresponding movement of the sensing probe assembly 66 relative to the housing 62. In this manner, the distal end 98 of the probe member 96 can be selectively moved out of the inner volume 76 of the housing 62 as desired, such as advancing the distal end 98 of the sensing probe 66 out of the housing 62 when the delivery device 60 has been coupled to the catheter assembly 12 and arterial blood monitoring and analysis is to be performed. As indicated above, in advancing the distal end 98 of the sensing probe 66 out of the housing 62 and into the catheter assembly 12, delivery device 60 may be configured to extend the distal end 98 of the sensing probe 66 out beyond the distal tip 26 of the indwelling arterial catheter 16.

Referring now to FIGS. 10 and 11. advancement of the sensor probe assembly 66 from the first, retracted position to the second, extended position is shown. The sensor probe assembly 66 can be in the first position prior to use and can be transitioned by a user (e.g., a doctor, physician, nurse, technician, phlebotomist, and/or the like) from the first position (FIG. 10) to the second position (FIG. 11) to dispose at least the distal end 98 of the probe member 96 in a distal position relative to the housing 62 (e.g., within the indwelling catheter 16 or distal to the catheter 16).

With the sensor probe assembly 66 disposed in the first position within the housing 62, substantially the entire probe member 96 is disposed within the housing 62. In other embodiments, the probe member 96 is disposed within the housing 62 and the lock 64 when sensor probe assembly 66 is in the first position.

The advancement member 68 is disposed in a proximal position when the sensor probe assembly 66 is in the first position, and the user may engage the tab 130 of the first portion 120 of the advancement member 68 to move the advancement member 68 relative to the housing 62, which in turn, moves the sensor probe assembly 66 from the first position (e.g., where probe member 96 is disposed within the housing 62) toward the second position. In this manner, the probe member 96 is moved out past the lock 64 and, as such, at least the distal end portion 98 of the probe member 96 is disposed outside of and distal to the lock 64.

The second position of the sensor probe assembly 66 is reached when the distal end portion 98 of the probe member 96 is placed in a desired position relative to a distal end 26 of the catheter 16 (see FIG. 1). In some instances, for example, a distal end 98 of the probe member 96 can be substantially flush with a distal end 26 of the catheter 16 when the sensor probe assembly 66 is in the second position. In other instances, the distal end 98 of the probe member 96 can extend a predetermined distance beyond the distal end 26 of the catheter 16, such that the distal end 98 of the probe member 96 is positioned within the artery (or other vein) at a predetermined distance beyond the distal end 26 of the catheter 16.

With the sensor probe assembly 66 in the second position (e.g., with the advancement member 68 advanced toward the distal end 74 of housing 62, for example, in FIG. 11), the connector portion 98 of sensor probe assembly 66 is brought adjacent to the distal end 74 of housing 62. In some embodiments, at least a portion of the connector portion 98—such as fitting 102 and flange 132 of connector portion 98—are positioned within coupler 78 of housing 62. The connector portion 98 may be sized and/or configured to be retained within coupler 78, such as via a press-fit or interference fit, for example, such that sensor probe assembly 66 may be secured relative to coupler 78 and lock 64.

With the sensor probe assembly 66 in the second position and the connector portion 98 secured within coupler 78 (and relative to lock 64), the detachable portion 80 of housing 62 may be disconnected. As described above and shown in FIGS. 4-6, detachable portion 80 of housing 62 may be actuated to disconnect from coupler 78, such as via a twist type disengagement of the detachable housing 80 or a separation and removal of detachable housing portions 80a, 80b. Thus, upon removal of the detachable housing 80, only the sensor probe assembly 66 and coupler 78 of delivery device 60 are left connected to and positioned adjacent catheter assembly 12.

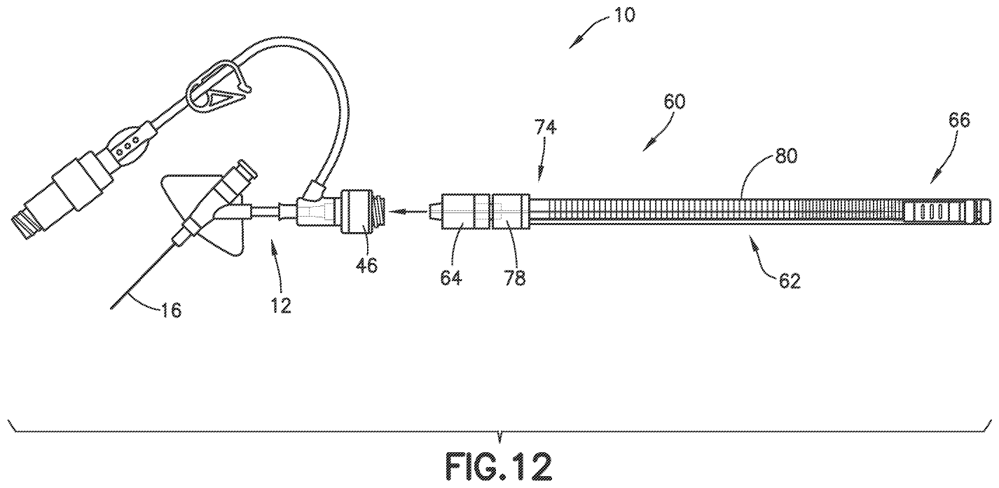

Referring now to FIGS. 12-14, a process flow (method) for interfacing a sensor probe delivery device 60 with a catheter assembly 12 for measuring and analyzing one or more blood-related parameters is provided. As first shown in FIG. 12, a sensor probe delivery device 60 is connected to an indwelling vascular access device (i.e., catheter assembly 12). In some embodiments, the sensor probe delivery device 60 is connected to an indwelling vascular access device via engaging of lock 64 with the near-patient access port provided by needleless access connector 46 of catheter assembly 12.

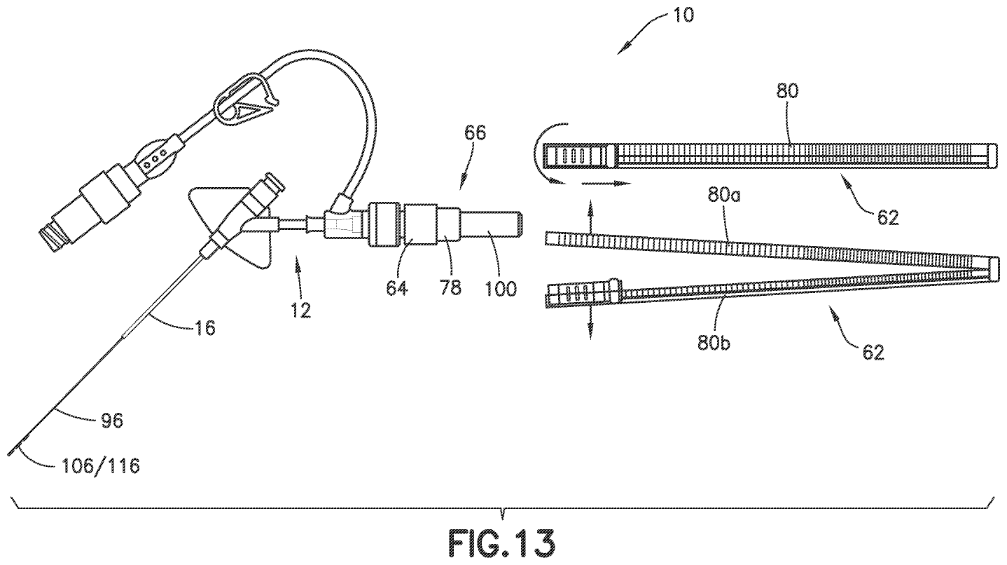

Upon connection of the sensor probe delivery device 60 to catheter assembly 12, the sensor probe delivery device 60 is operated to advance the probe member 96 (with sensor(s) 106, 116, for example) into the fluid path of indwelling catheter 16 or beyond the distal tip 26 thereof. In advancing the sensor probe assembly 66, the advancement member 68 is moved distally relative to the housing 62, which in turn, moves the sensor probe assembly 66 from a first position where probe member 96 is disposed within the housing 62 to a second position where a distal end 98 of the probe member 96 is disposed into the fluid path of indwelling catheter 16 or beyond the distal tip 26 thereof. In advancing the sensor probe assembly 66 to the second position, a connector portion 98 of the sensor probe assembly 66 is brought to the distal end 74 of housing 62 (e.g., within coupler 78) and adjacent to lock 64. With the sensor probe assembly 66 in this position, the detachable housing 80 is disconnected from coupler 78 (and lock 64) as shown in FIG. 13, such that only the sensor probe assembly 66 of delivery device 60 remains connected to catheter assembly 12.

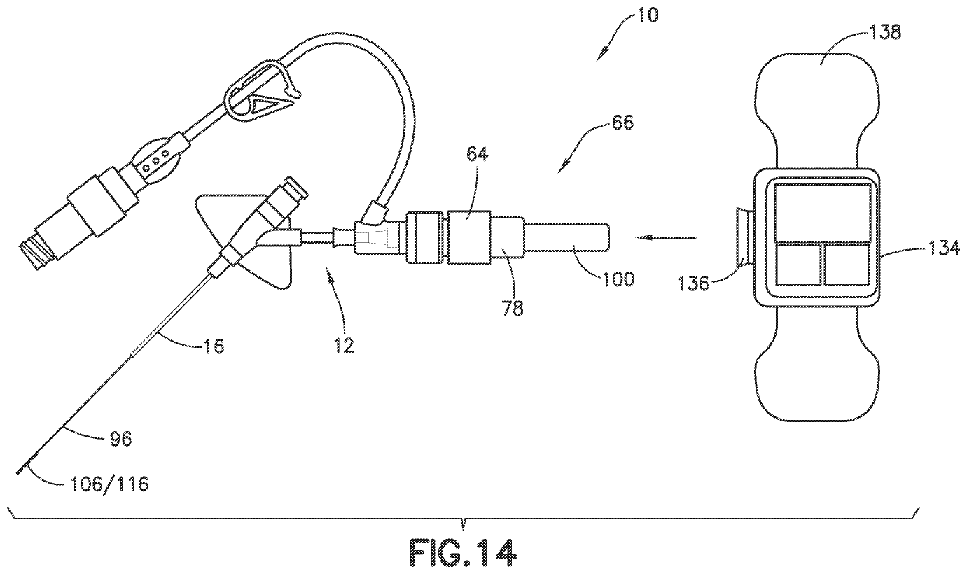

Upon removal of the detachable housing 80 from sensor probe assembly 66, access to the electrical connector 100 of sensor probe assembly 66 is provided. In accordance with some aspects of the disclosure, a wireless module 134 may be coupled to the electrical connector 100 of sensor probe assembly 66, as shown in FIG. 14. In the illustrated embodiment, wireless module 134 is provided as a wireless board unit 134 that may include a processor, communications, power source, sensor leads, and user interface, as explained further below. The wireless board unit 134 may be connected to electrical connector 100 of sensor probe assembly 66 via an input connection 136 provided thereon. Upon connection to sensor probe assembly 66, the wireless board unit 134 may be placed/secured against the skin of a patient, such as via a stabilization platform 138 provided on the unit and/or via a securement dressing or other securement method.

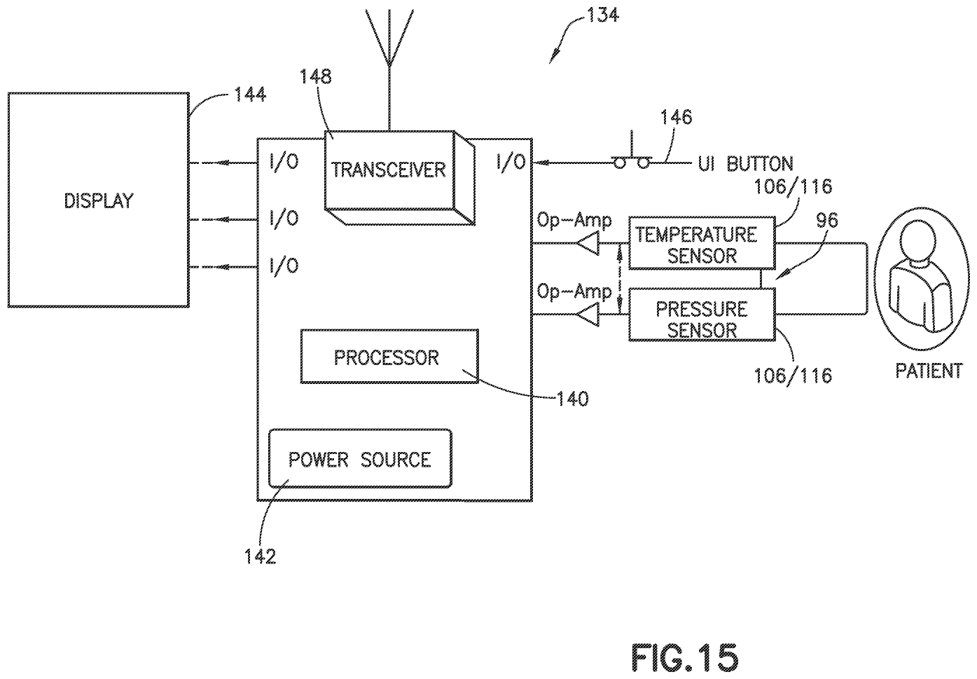

As shown in FIG. 15, the wireless board unit 134 may include a processor 140, a power source 142, a display 144, a user interface 146, and a radio transceiver 148 configured to transmit data from the sensor(s) 106, 116 wirelessly, such as through a Bluetooth connection, to various external devices. The processor 140, display 144, user interface 146, and radio transceiver 148 of the wireless board unit 134 may be provided on a SoC microcontroller, although other suitable configurations may be utilized. The display 144 of wireless board unit 134 may be configured to display measured blood-related parameter data thereon and/or indicate any alerts to a user, while the user interface 146 may include thereon a button or other suitable feature via which a user may provide commands to the unit 134.

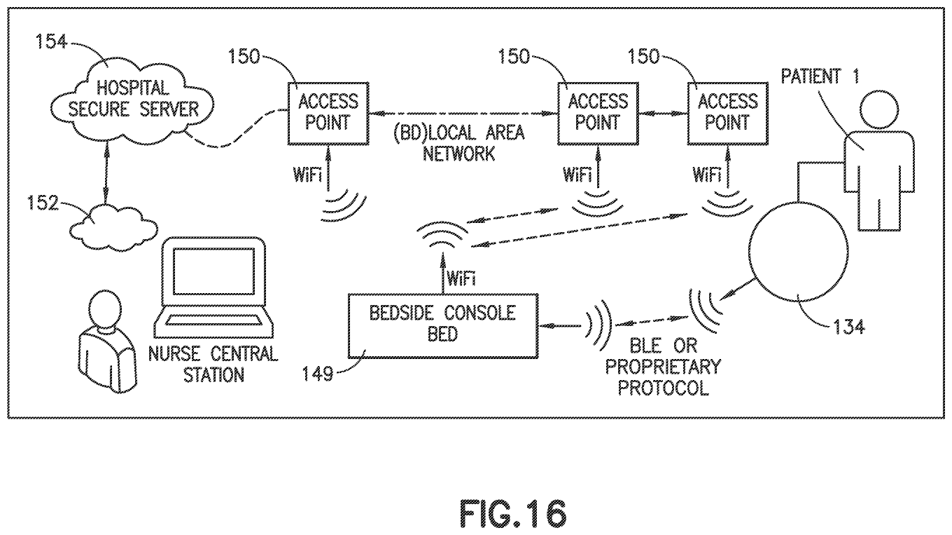

Upon connection of the wireless board unit 134 to sensor probe assembly 66, the wireless board unit 134 may receive measurements of one or more blood-related parameters from sensors (sensor(s) 106, 116, for example) on probe member 96 and subsequently analyze and/or further communicate these measurements to another processing device. In one aspect or embodiment, as illustrated in FIG. 16, the wireless board unit 134 transmits data from the second probe assembly 66 to a bedside console 149, which, in turn, transmits the data to a local area network via Wi-Fi access points 150 or another suitable network connection. The local area network may be connected to the cloud 152 and/or secure hospital server 154, which can be accessed at a nurse central station 102 or other remote location. The wireless board unit 134 may thus provide a mobile data capture, analysis, and monitoring unit that may be used to provide blood-related parameter assessment at the bedside, at a nurse central station 102, or at a remote data monitoring and analysis center. Via the wireless communication capability provided thereby, the wireless board unit 134 may feed data measurements directly to the cloud for monitoring, processing, analysis, and artificial intelligence (AI) predictive analysis related to the acquired one or more blood-related parameters and/or for transferring to the patient's electronic medical record or to a nurse monitoring station system or other patient monitoring displays and systems. In some embodiments, the wireless board unit 134 may also alert a clinician, technician or patient, if a measured blood-related parameter is determined to be at a critical level (such as via analysis by AI algorithm(s) run by the unit or a remote data monitoring and analysis center)—with such alerts provided as visual, audible, tactile and/or digital alerts or indicators on a visual display or a system component (wired or wireless connection). In still other embodiments, data, images, or alerts may be captured or communicated via other various systems and displays, including infusion pumps, patient vital sign monitors, arterial monitors, ultrasound system visual display, smart phone, tablets, PCs or other system, with such outputs being combined (at a nurse station or at a remote data monitoring and analysis center) with the one or more blood-related parameters acquired by the sensor probe assembly 66 for a more comprehensive monitoring and analysis of patient physiological data.

Upon completion of data acquisition and analysis of the one or more blood-related parameters, the probe member 96 may be removed/withdrawn from the indwelling catheter, and the sensor probe assembly 66 may be disconnected from catheter assembly 12, such as via disengaging of lock 64 from needleless access connector 46.

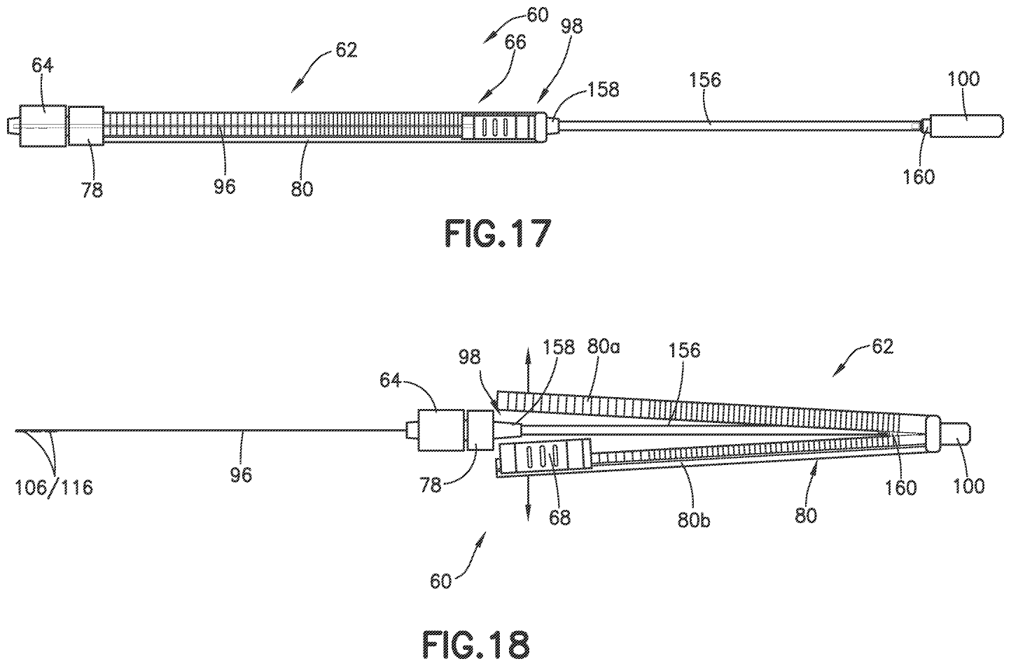

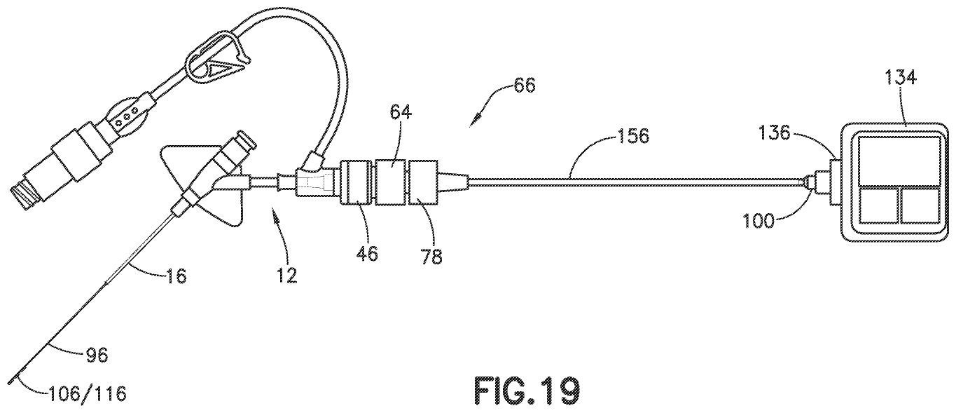

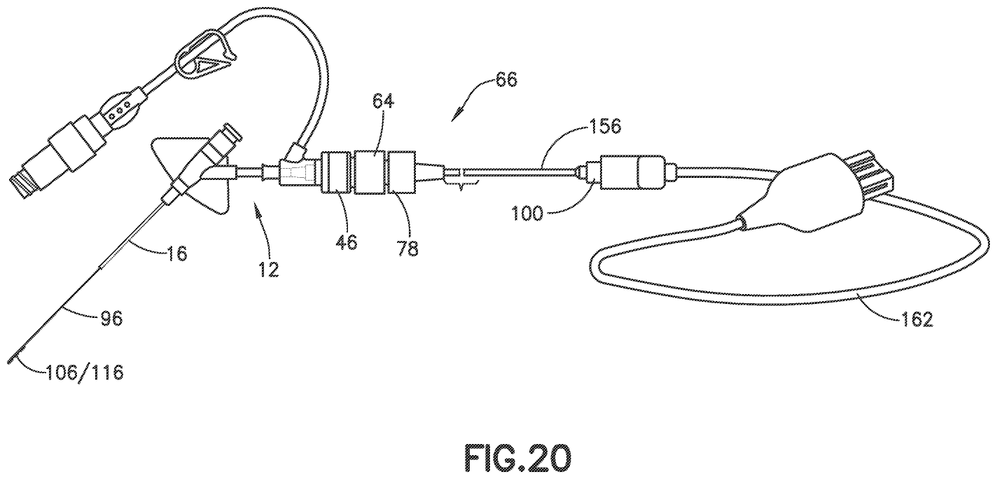

According to some aspects of the disclosure, the sensor probe delivery device 60 may be configured to locate the electrical connector 100 of sensor probe assembly 66 further away from the insertion site of catheter assembly 12. That is, as shown in FIGS. 17-20, the sensor probe delivery device 60 may include a wired extension lead or tether 156 (hereafter “wired extension 156”) that is provided as part of sensor probe assembly 66, with the wired extension 156 provided between the probe member 96 and electrical connector 100. The wired extension 156 may include a first fitting 158 at the distal end thereof that connects the wired extension 156 to connector portion 98 of the sensor probe assembly 66, and a second fitting 160 at the proximal end thereof that connects the wired extension 156 to electrical connector 100. In the initial configuration of sensor probe delivery device 60, the wired extension 156 may be positioned outside of (and proximal to) the housing 62 of delivery device 60 (FIG. 17). In actuating the advancement member 68 of delivery device 60 in a distal direction, the sensor probe assembly 66 is also moved distally (based. for example, on coupling of advancement member 68 to connection portion 98), with the probe member 96 thus moved into the catheter assembly 12 and the wired extension 156 moved into the housing 62 (FIG. 18). Upon disconnection of the detachable housing 80, the sensor probe assembly 66 is thus in position for a subsequent vasculature monitoring and analysis to be performed, with the electrical connector 100 at the proximal end of wired extension 156 enabling connection of an external device to the sensor probe assembly 66. In the embodiment of FIG. 19, a wireless board unit 134 (as previously described) is connected to the electrical connector 100 at the proximal end of wired extension 156—with the wired extension 156 allowing remote securement of the wireless board unit 134 (i.e., remote from an insertion site). In the embodiment of FIG. 20, a patient data cable 162 is connected to the electrical connector 100 at the proximal end of wired extension 156, with the data cable 162 connectable to a nurse monitoring station system or other patient monitoring display and system that may provide blood-related parameter assessment of the data acquired via sensor probe assembly 66.

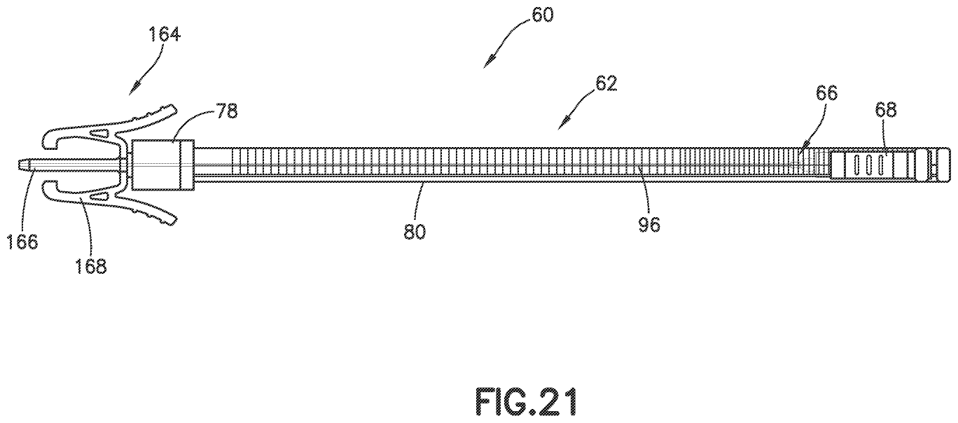

While exemplary embodiments of sensor probe delivery device 60 for use in an arterial access system 10 have been described above, it is recognized that variations to the device may be made according to additional aspects of the disclosure. As one example, and as illustrated in FIG. 21, the sensor probe delivery device 60 may include a lock 164 structured differently from that shown and described in the embodiment of FIGS. 1-11. That is, a lock 164 may be provided in sensor probe delivery device 60 that includes a blunted cannula 166 and locking arms 168 for coupling to the needleless access connector 46 of catheter assembly 12, with the blunted cannula 166 and locking arms 168 forming three points of contact therewith.

Other variations to the sensor probe delivery device 60 previously shown and described may include alterations to the type of advancement member 68 included therein and/or to the sensor probe assembly 66—such as the sensor probe assembly 66 being configured as a mixed wireless and wired system, with a wireless board unit 134 measuring certain parameters and a wired connection measuring other parameters that require a higher power connection to monitor.

Beneficially, embodiments of the disclosure thus provide a sensor probe delivery device that enables positioning of a probe member (with sensor(s) thereon) within an indwelling catheter. The delivery device include a detachable housing portion that may be removed, such that a sensor probe assembly may remain in place long term adjacent a catheter insertion site, while the sensor probe assembly is less bulky and more patient friendly for long term sensing requirements. Removal of the detachable housing minimizes the footprint of the sensor probe assembly and reduces the risk of dislodgement or other complications that may arise from a bulky rigid device remaining attached to a patient for a longer period.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A vascular access system comprising:
a catheter assembly comprising a catheter and a near-patient access port; and
a sensor probe delivery device coupleable to the near-patient access port to enable access to the catheter via the catheter assembly, the delivery device comprising:
a sensing probe assembly comprising:
a probe member including one or more sensors configured to measure one or more blood-related parameters, the probe member having a distal end and a proximal end; and
an electrical connector positioned proximally from the probe member, the electrical connector configured to operably connect to an external device;
a housing configured to movably receive at least a portion of the sensing probe assembly within an inner volume thereof, the housing comprising a detachable portion separable from a remainder of the housing;
a lock configured to couple the housing to the near patient access port; and
an advancement member configured to move relative to the housing to move the sensing probe assembly between a first position, in which a distal end of the probe member is disposed within the housing or the lock, and a second position in which a distal end of the probe member is disposed beyond the distal end portion of the housing and the lock and into the catheter or out past a distal tip of the catheter;
wherein, with the sensing probe assembly in the second position, the probe member is positioned to measure the one or more blood-related parameters.

2. The vascular access system of claim 1, wherein the housing comprises a coupler provided at a distal end portion thereof, the coupler configured to mate with the lock or formed integrally with a portion of the lock, and wherein the detachable portion of the housing is configured to disconnect from the coupler.

3. The vascular access system of claim 2, wherein the detachable portion of the housing is configured to disconnect from the coupler via a twist-type disconnection.

4. The vascular access system of claim 2, wherein the detachable portion comprises a splittable housing including a pair of detachable housing portions that separate off from the coupler.

5. The vascular access system of claim 2, wherein the sensing probe assembly comprises a connector portion including:
a fitting configured to secure the proximal end of the probe member therein; and
a flange member joined with the fitting;
wherein the connector portion is positioned adjacent the coupler when the sensing probe assembly is in the second position, with the connector portion retained in the coupler.

6. The vascular access system of claim 5, wherein the advancement member is coupled with the flange of the connector portion, such that movement of the advancement member relative to the housing causing a corresponding movement of the sensing probe assembly.

7. The vascular access system of claim 5, wherein the electrical connector is joined to the connector portion and extends out proximally therefrom.

8. The vascular access system of claim 7, wherein the electrical connector is positioned within the housing when the sensing probe assembly is in each of the first position and the second position, with the electrical connector being accessible when the sensing probe assembly is in the second, upon disconnecting of the detachable portion.

9. The vascular access system of claim 5, wherein the sensing probe assembly comprises a wired extension joined to the connector portion and extending out proximally therefrom, and wherein the electrical connector is coupled to the wired extension at a proximal end thereof.

10. The vascular access system of claim 9, wherein with the electrical connector coupled to the proximal end of the wired extension, the electrical connector is spaced apart from the connector portion and the coupler when the sensing probe assembly is in the second position.

11. The vascular access system of claim 1, further comprising a wireless module coupled to the electrical connector, the wireless module configured to:
receive the one or more blood-related parameters from the sensing probe assembly; and
analyze and display the one or more blood-related parameters and/or wirelessly transmit the one or more blood-related parameters to a processing device.

12. The vascular access system of claim 1, further comprising a patient data cable coupled to the electrical connector and configured to transmit the one or more blood-related parameters to a processing device.

13. The vascular access system of claim 1, wherein the catheter assembly comprises:
a catheter adapter coupled to a proximal end of the catheter, the catheter adapter comprising an adapter port; and
a connector connected to the adapter port via an extension tube, wherein the near patient access port is provided at a proximal end of the connector.

14. A method of using a vascular access system, the method comprising:
providing a vascular access system having a catheter assembly including a catheter and a near-patient access port, and a sensor probe delivery device coupleable to the near-patient access port to enable access to the catheter via the catheter assembly, the delivery device including a sensing probe assembly having a probe member including one or more sensors configured to measure one or more blood-related parameters, the probe member having a distal end and a proximal end, and an electrical connector positioned proximally from the probe member, the electrical connector configured to operably connect to an external device, the vascular access system also including a housing configured to movably receive at least a portion of the sensing probe assembly within an inner volume thereof, the housing comprising a detachable portion separable from a remainder of the housing, a lock configured to couple the housing to the near patient access port, and an advancement member configured to move relative to the housing to move the sensing probe assembly between a first position, in which a distal end of the probe member is disposed within the housing or the lock, and a second position in which a distal end of the probe member is disposed beyond the distal end portion of the housing and the lock and into the catheter or out past a distal tip of the catheter; with the sensing probe assembly in the second position, the probe member is positioned to measure the one or more blood-related parameters;
coupling the sensor probe delivery device to the near-patient access port of the catheter assembly;
advancing the sensor probe assembly from the first position to the second position such that the distal end of the probe member extends into the catheter or beyond the distal end of the catheter;
disconnecting the detachable portion of the housing; and
connecting an external device to the electrical connector of the sensor probe assembly, the external device comprising one of a wireless module and a data cable.

15. The method of claim 14, comprising securing the wireless module in place via one or more of a stabilizing platform and a securement dressing.

16. A sensor probe delivery device coupleable to a near-patient access port of a catheter assembly, the delivery device comprising:
a sensing probe assembly comprising:
a probe member including one or more sensors configured to measure one or more blood-related parameters, the probe member having a distal end and a proximal end; and
an electrical connector positioned proximally from the probe member, the electrical connector configured to operably connect to an external device;
a housing configured to movably receive at least a portion of the sensing probe assembly within an inner volume thereof, the housing comprising a detachable portion separable from a remainder of the housing;
a lock configured to configured to be connected to the near patient access port; and
an advancement member configured to move relative to the housing to move the sensing probe assembly between a first position, in which a distal end of the probe member is disposed within the housing or the lock, and a second position in which a distal end of the probe member is disposed beyond the distal end portion of the housing and the lock;
wherein, with the sensing probe assembly in the second position, the probe member is positioned to measure the one or more blood-related parameters.

17. The sensor probe delivery device of claim 16, wherein the housing comprises a coupler provided at a distal end portion thereof, the coupler configured to mate with the lock or formed integrally with a portion of the lock, and wherein the detachable portion of the housing is configured to disconnect from the coupler.

18. The sensor probe delivery device of claim 17, wherein the sensing probe assembly comprises a connector portion including:
a fitting configured to secure the proximal end of the probe member therein; and
a flange member joined with the fitting;
wherein the connector portion is positioned adjacent the coupler when the sensing probe assembly is in the second position, with the connector portion retained in the coupler.

19. The sensor probe delivery device of claim 18, wherein the advancement member is coupled with the flange of the connector portion, such that movement of the advancement member relative to the housing causing a corresponding movement of the sensing probe assembly.

20. The sensor probe delivery device of claim 18, wherein the electrical connector is joined to the connector portion and extends out proximally therefrom, with the electrical connector positioned within the housing when the sensing probe assembly is in each of the first position and the second position, and with the electrical connector being accessible when the sensing probe assembly is in the second, upon disconnecting of the detachable portion.
